SEQUENCE LISTING (17 sequences)

SEQ ID NO: 1 - protein, 16 aa, artificial
  synthetically generated polypeptide
CLGTSKLNYLDPRITV

SEQ ID NO: 2 - protein, 16 aa, artificial
  synthetically generated polypeptide
CLGTSKLNYLDPRITV

SEQ ID NO: 3 - protein, 16 aa, artificial
  synthetically generated polypeptide
CXXXXXXXYXXXXXXX

SEQ ID NO: 4 - protein, 16 aa, artificial
  synthetically generated polypeptide
CKDSASPRYIFTMLSS

SEQ ID NO: 5 - protein, 16 aa, artificial
  synthetically generated polypeptide
CKDAASPRYIFTMLST

SEQ ID NO: 6 - protein, 16 aa, artificial
  synthetically generated polypeptide
CKDSASPRYIFTMLSS

SEQ ID NO: 7 - protein, 16 aa, artificial
  synthetically generated polypeptide
CKDAASPRYIFTMLST

SEQ ID NO: 8 - protein, 16 aa, artificial
  synthetically generated polypeptide
CLGTSKLNYLDPRITV

SEQ ID NO: 9 - protein, 16 aa, artificial
  synthetically generated polypeptide
CLGTSKLNYLDPRITV

SEQ ID NO: 10 - protein, 16 aa, artificial
  synthetically generated polypeptide
CLGTSKLNYLDPRITV

SEQ ID NO: 11 - protein, 15 aa, artificial
  synthetically generated polypeptide
LGTSKLNYLDPRITV

SEQ ID NO: 12 - protein, 15 aa, artificial
  synthetically generated polypeptide
LGTSKLNYLDPRITV

SEQ ID NO: 13 - protein, 15 aa, artificial
  synthetically generated polypeptide
XXXXXXXYXXXXXXX

SEQ ID NO: 14 - protein, 15 aa, artificial
  synthetically generated polypeptide
KDSASPRYIFTMLSS

SEQ ID NO: 15 - protein, 15 aa, artificial
  synthetically generated polypeptide
KDAASPRYIFTMLST

SEQ ID NO: 16 - protein, 15 aa, artificial
  synthetically generated polypeptide
KDSASPRYIFTMLSS

SEQ ID NO: 17 - protein, 15 aa, artificial
  synthetically generated polypeptide
KDAASPRYIFTMLST

(19) United States
(12) Patent Application Publication
(10) Pub. No.: US 2017/0003289 A1
(43) Pub. Date: Jan. 5, 2017

(54) METHODS AND MATERIALS FOR ASSESSING ENZYME-NUCLEIC ACID COMPLEXES

(71) Applicants: REGENTS OF THE UNIVERSITY OF MINNESOTA, Minneapolis, MN (US); Mayo Foundation for Medical Education and Research, Rochester, MN (US)

(72) Inventors: Daniel A. Harki, Minneapolis, MN (US); Scott H. Kaufmann, Rochester, MN (US); Angela Lyn Perkins-Harki, Minneapolis, MN (US)

(21) Appl. No.: 15/198,823

(22) Filed: Jun. 30, 2016

Related U.S. Application Data
(60) Provisional application No. 62/186,816, filed on Jun. 30, 2015.

(51) Int. Cl. G01N 33/573; C07K 16/44; G01N 33/53; C07K 16/40

(52) U.S. Cl. CPC G01N 33/573; C07K 16/40; C12Y 599/01002; C07K 16/44; G01N 33/5308; C07K 2317/92; G01N 2333/99; G01N 2800/52

(57) ABSTRACT

Materials and methods for assessing the presence, absence, and/or amount of an enzyme-nucleic acid complex in a sample are provided. Also provided are materials and methods for making and using an anti-enzyme-nucleic acid covalent complex antibody.

### CONTINUING APPLICATION DATA

This application claims the benefit of U.S. Provisional Application Ser. No. 62/186,816, filed Jun. 30, 2015, which is incorporated by reference herein.

### SEQUENCE LISTING

This application contains a Sequence Listing electronically submitted to the United States Patent and Trademark Office via EFS-Web as an ASCII text file entitled “110-04980101-sequencelisting_ST25.txt” having a size of 6 kilobytes and created on Jun. 30, 2016. Due to the electronic filing of the Sequence Listing, the electronically submitted Sequence Listing serves as both the paper copy required by 37 CFR §1.821(c) and the CRF required by §1.821(e). The information contained in the Sequence Listing is incorporated by reference herein.

### SUMMARY OF THE INVENTION

Enzymes that are present in both prokaryotes and eukaryotes regulate the overwinding or underwinding of nucleic acids, particularly during replication and transcription. Drugs, including, for example, members of a widely used class of anticancer drugs, the camptothecins, can interfere with these enzymes, resulting in increased formation of covalent enzyme-DNA complexes. These covalent enzyme-DNA complexes can drive DNA damage, ultimately leading to cell death. Assessing the presence, absence, and/or amount of enzyme-nucleic acid complexes in a sample before and/or after a drug treatment designed to target an enzyme that forms a covalent bond with nucleic acid can allow clinicians and other medical professionals to monitor the effectiveness of that particular treatment.

In one aspect, this disclosure describes an isolated antibody preparation including an anti-enzyme-nucleic acid covalent complex antibody. The anti-enzyme-nucleic acid covalent complex antibody binds to an enzyme-nucleic acid covalent complex and does not bind to the free form of the enzyme or the free form of the nucleic acid. In some embodiments, the anti-enzyme-nucleic acid covalent complex antibody is produced using an antigen that includes a polypeptide and a nucleoside. In some embodiments, the anti-enzyme-nucleic acid covalent complex antibody binds to an antigen that includes a polypeptide and a nucleoside. The polypeptide and the nucleoside are covalently complexed.

In some embodiments, the nucleic acid is DNA. In some embodiments, the polypeptide includes a phosphorylated tyrosine, and the nucleoside is covalently complexed to the phosphorylated tyrosine. In some embodiments, the polypeptide includes the amino acid sequence set forth in SEQ ID NO:1, SEQ ID NO:4, SEQ ID NO:5, SEQ ID NO:11, SEQ ID NO:13, or SEQ ID NO:14. In some embodiments, the nucleoside includes at least one of cytidine, deoxycytidine, uridine, deoxyuridine, adenosine, deoxyadenosine, guanosine, deoxyguanosine, thymidine, or deoxythymidine. In some embodiments, the enzyme includes a topoisomerase.

In some embodiments, the anti-enzyme-nucleic acid covalent complex antibody is produced using a polypeptide antigen comprising the amino acid sequence set forth in SEQ ID NO:1, SEQ ID NO:4, SEQ ID NO:5.

In some embodiments, the antigen includes

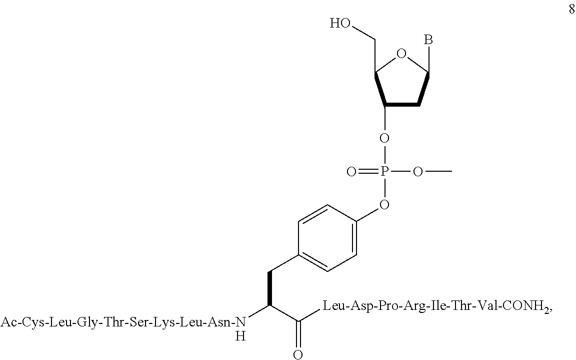

wherein B comprises a nucleobase, or

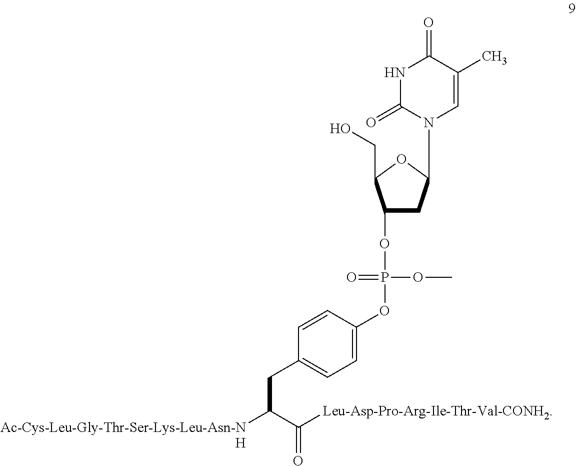

In some embodiments, the anti-enzyme-nucleic acid covalent complex antibody is produced using a polypeptide antigen comprising

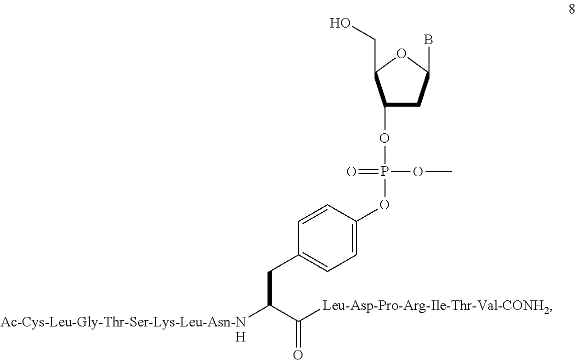

wherein B comprises a nucleobase, or

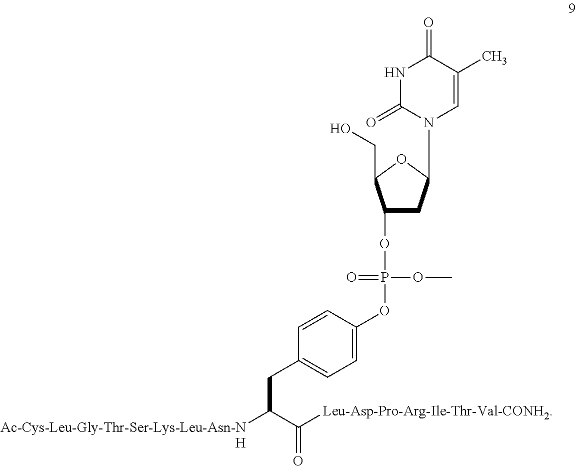

This disclosure further describes an isolated antibody preparation including an anti-enzyme-nucleic acid covalent complex antibody. The anti-enzyme-nucleic acid covalent complex antibody binds to an enzyme-nucleic acid covalent complex and does not bind to the free form of the enzyme or the free form of the nucleic acid.

In some embodiments, the K_{D} of the anti-enzyme-nucleic acid covalent complex antibody and an enzyme-nucleic acid covalent complex is no greater than 1×10⁻⁸, no greater than 1×10⁻⁹, no greater than 1×10⁻¹⁰, no greater than 1×10⁻¹¹, or no greater than 1×10⁻¹².

In another aspect, this disclosure describes a method for assessing a sample for enzyme-nucleic acid covalent complexes. The method includes performing an immune-based assay using an anti-enzyme-nucleic acid covalent complex antibody to detect the presence, absence, or amount of the enzyme-nucleic acid covalent complexes within the sample. The anti-enzyme-nucleic acid covalent complex antibody binds to the enzyme-nucleic acid covalent complex and does not bind to the free form of the enzyme or the free form of the nucleic acid. The K_{D} of the anti-enzyme-nucleic acid covalent complex antibody and the enzyme-nucleic acid covalent complex is no greater than 1×10⁻⁸, no greater than 1×10⁻⁹, no greater than 1×10⁻¹⁰, no greater than 1×10⁻¹¹, or no greater than 1×10⁻¹². In some embodiments, the sample includes a cell or a tissue sample. In some embodiments, the sample is obtained from a subject following administration to the subject of a drug treatment designed to result in the accumulation of enzyme-nucleic acid covalent complexes within cells, and the anti-enzyme-nucleic acid covalent complex antibody is able to detect the presence of the enzyme-nucleic acid covalent complexes within the sample.

In a further aspect, this disclosure describes a method for monitoring the effectiveness of a drug treatment designed to result in the accumulation of enzyme-nucleic acid covalent complexes within cells. The method includes performing an immune-based assay using an anti-enzyme nucleic acid covalent complex antibody to detect the presence or absence of the enzyme-nucleic acid covalent complexes within a sample obtained from a mammal following treatment of the mammal with the drug treatment. In some embodiments, the drug treatment preferably includes treatment with a drug designed to stabilize an enzyme-nucleic acid covalent complex. The anti-enzyme-nucleic acid covalent complex antibody binds to the nucleic acid covalent complex and does not bind to the free form of the enzyme, a peptide fragment of the free form of the enzyme, and/or a free form of the nucleic acid. In some embodiments, the K_{D} of the anti-enzyme-nucleic acid covalent complex antibody and the enzyme-nucleic acid covalent complex is no greater than 1×10⁻⁸, no greater than 1×10⁻⁹, no greater than 1×10⁻¹⁰, no greater than 1×10⁻¹¹, or no greater than 1×10¹². In some embodiments, the K_{D} of the anti-enzyme-nucleic acid covalent complex antibody and the enzyme-nucleic acid covalent complex is greater than the K_{D} of the anti-enzyme-nucleic acid covalent complex antibody and the free form of the enzyme and/or greater than the K_{D} of the anti-enzyme-nucleic acid covalent complex antibody and a peptide fragment of the free form of the enzyme.

In some embodiments, the drug treatment is a topoisomerase I inhibitor or poison. In some embodiments, the drug treatment is an irinotecan, topotecan, or camptothecin treatment. In some embodiments, the drug treatment is a topoisomerase II inhibitor or poison.

In another aspect, this disclosure describes a composition including a polypeptide and a nucleoside. The polypeptide includes the amino acid sequence of an active site of an enzyme that binds to DNA. The polypeptide and the nucleoside are covalently complexed via a phosphate group. In some embodiments, the polypeptide and the nucleoside are covalently complexed via a phosphate linkage to a 5′-alcohol or a 3′-alcohol.

In some embodiments, the polypeptide includes the amino acid sequence set forth in SEQ ID NO:1, SEQ ID NO:4, SEQ ID NO:5, SEQ ID NO:11, SEQ ID NO:13, or SEQ ID NO:14. In some embodiments, the nucleoside is cytidine, deoxycytidine, uridine, deoxyuridine, adenosine, deoxyadenosine, guanosine, deoxyguanosine, thymidine, or deoxythymidine. In some embodiments, the enzyme that binds to DNA is topoisomerase I or topoisomerase II. In some embodiments, the polypeptide includes phosphorylated tyrosine, and the nucleoside is covalently complexed to the phosphorylated tyrosine.

In another aspect, this disclosure describes a composition including

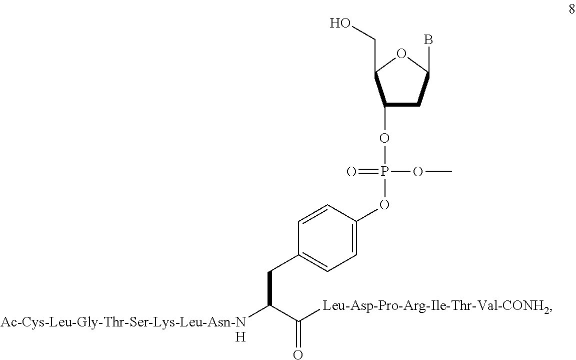

wherein B comprises a nucleobase.

In a further aspect, this disclosure describes a composition including

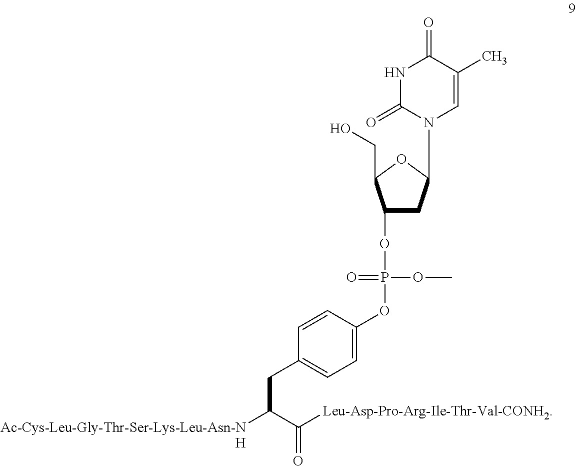

This disclosure also describes a method of making the compositions described herein.

An “antibody” and “antibodies” (immunoglobulins) refer to at least one of a monoclonal antibody (including a full-length monoclonal antibody), a polyclonal antibody preparation, a multispecific antibody (e.g., bispecific antibodies) formed from at least two intact antibodies, a human antibody, a humanized antibody, a camelized antibody, a chimeric antibody, a single-chain variable fragment (scFv), a single-chain antibody, a single domain antibody, a domain antibody, an antibody fragment including, without limitation, an Fab fragment, an F(ab′)₂ fragment, an antibody fragment that exhibits the desired biological activity, a disulfide-linked Fv (sdFv), an intrabody, or an epitope-binding fragment of any of the above. In particular, antibody includes an immunoglobulin molecule and an immunologically active fragment of an immunoglobulin molecule, i.e., a molecule that contains an antigen-binding site. Immunoglobulin molecules can be of any type e.g. IgG, IgE, IgM, IgD, IgA and IgY), class (e.g., IgG1, IgG2, IgG3, IgG4, IgA1 and IgA2) or subclass.

“Epitope” refers to a chemical moiety that exhibits specific binding to an antibody.

“Specific” and variations thereof refer to having a differential or a non-general affinity, to any degree, for a particular target.

The terms “comprises” and variations thereof do not have a limiting meaning where these terms appear in the description and claims.

Unless otherwise specified, “a,” “an,” “the,” and “at least one” are used interchangeably and mean one or more than one.

Also herein, the recitations of numerical ranges by endpoints include all numbers subsumed within that range (e.g., 1 to 5 includes 1, 1.5, 2, 2.75, 3, 3.80, 4, 5, etc.).

For any method disclosed herein that includes discrete steps, the steps may be conducted in any feasible order. And, as appropriate, any combination of two or more steps may be conducted simultaneously.

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1 shows a schematic representation of the formation of topoisomerase I-DNA covalent complex; B=nucleobase; Topo I=topoisomerase I.

FIG. 2 shows a first-generation peptide-nucleotide antigen 1 (SEQ ID NO:1) and second-generation peptide-nucleotide antigen 2 (SEQ ID NO:8).

FIG. 3 shows a scheme for the synthesis of monomer for solid-phase peptide synthesis. Reagents and Conditions: a) i. allyl bromide, DIPEA, DMF; ii. CF₃CO₂H in CH₂Cl₂ (1:49), yield: 74% (2 steps); b) tetrazole (0.47 M) in CH₂Cl₂, 4 Å mol. sieves, yield: 69%; c) i. t-BuOOH (5-6 M in decane), CH₂Cl₂; ii. Pd(PPh₃)₄, PhSiH₃, THF, yield: 60% (2 steps).

FIG. 4 shows analytical characterization of antigen 2 by reverse-phase HPLC and mass spectrometry.

FIG. 5 shows peptide-nucleotide antigen 8 (SEQ ID NO:9); B=nucleobase.

FIG. 6 shows peptide-nucleotide antigen 9 (SEQ ID NO:10).

FIG. 7 shows A, concentrated culture supernatants from the four indicated hybridomas and normal mouse serum that were subjected to ELISA assay using peptide-nucleobase conjugate (antigen 2) or unphosphorylated topo I active site peptide (SEQ ID NO 2). B, reactivity of 6F9 supernatant, an antibody raised against peptide-nucleotide antigen 1 (SEQ ID NO 1) or an unrelated monoclonal antibody (raised against the signaling protein RasGRP1) with peptide-nucleobase conjugate (antigen 2), phosphorylated topo I active site peptide 1 (SEQ ID NO 1), or unphosphorylated topo I active site peptide (SEQ ID NO 2). C, reactivity of serially diluted 6F9 supernatant with peptide-nucleobase conjugate (antigen 2), phosphorylated topo I active site peptide 1 (SEQ ID NO 1), or unphosphorylated topo I active site peptide (SEQ ID NO 2).

### DETAILED DESCRIPTION

This disclosure provides materials and methods for assessing the presence, absence, and/or amount of an enzyme-nucleic acid complex in a sample. Assessing the presence, absence, and/or amount of an enzyme-nucleic acid complex in a sample before and/or after a drug treatment designed to affect an enzyme-nucleic acid complex can allow clinicians and other medical professionals to monitor the effectiveness of that drug treatment. Accurate monitoring of the effectiveness involves an assessment method that is sufficiently sensitive to detect the enzyme-nucleic acid complex in the amounts formed after drug treatment.

This disclosure provides antibodies; antibody preparations; hybridomas; methods for making antibodies; antigens that may be used to generate an antibody; methods for making antigens; methods for using antibodies to determine the presence, absence, and/or amount of an enzyme-nucleic acid complex; and methods for monitoring the effectiveness of a drug treatment designed to affect the accumulation of enzyme-nucleic acid complexes in cells.

### Enzyme Nucleic Acid Covalent Complexes, Topoisomerases, and Anti-Cancer Drugs

A number of enzymes form covalent bonds with nucleic acid (e.g., DNA). Such enzymes include, without limitation, topoisomerase I (topo I), topoisomerase II (topo II), topoisomerase III (topo III), tyrosyl deoxynucleotidyl phosphodiesterase (TDP1), and yeast, protozoal, or bacterial topoisomerases (e.g., DNA gyrase, topoisomerase IV, and topoisomerase VI).

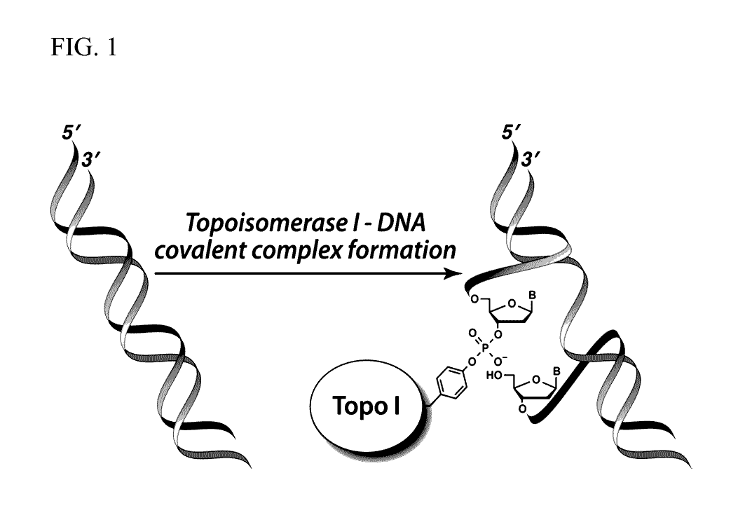

Both topo I and topo II control DNA supercoiling. Human topo I is abundant in the cell (up to 10⁶ copies/cell) and relaxes torsional strain for a variety of nuclear processes, including replication, transcription, and viral integration. During the course of its normal catalytic cycle, topo I catalyzes a transesterification reaction that results in the formation of a covalent bond between the active site tyrosine of the enzyme and a 3′-phosphate in the DNA backbone, with concomitant formation of a nick in the DNA backbone that allows rotation of DNA around the intact strand (FIG. 1). After the DNA is relaxed, the enzyme reverses the transesterification reaction, thereby resealing the DNA backbone and restoring DNA integrity. During the course of its normal catalytic cycle, topo II catalyzes a transesterification reaction that results in the formation of a covalent bond between an active site tyrosine of the enzyme and a 5′-phosphate in both strands of the DNA backbone.

A number of approved and investigational anti-cancer drugs target one or more enzymes that form covalent bonds with nucleic acid. For example, camptothecin and its derivatives target topo I. These drugs may induce cytotoxicity by preventing the religation step of topo I, resulting in an increase in the number of covalent topo I-DNA complexes. Interactions between the enzyme-DNA complexes with replication forks and transcription complexes can result in DNA damage, ultimately leading to cell death.

For example, nalidixic acid, ciprofloxacin and its derivatives, novobiocin, etoposide, etoposide phosphate, teniposide, daunorubcin, doxorubicin, mitoxantrone, ICRF-187 (dexraxozane), amsacrine, ellipticinium, TAS-103, genistein, and CP-115,953 target topo II. These drugs may induce cell death by one of several mechanisms including stabilizing Topo II-DNA cleavage complexes to yield DNA strand breaks, interfering with breaking and rejoining of DNA, enhancing the formation of topo II-DNA covalent complexes, and/or inhibiting ATP hydrolysis of topo II.

Based on their ability to selectively induce cancer cell death, several topo I- and topo II-targeting drugs are anticancer agents. For example, irinotecan is FDA-approved for colorectal cancer and is active against non-small cell lung cancer, pancreatic cancer, and breast cancer, and topotecan (TPT) is approved for ovarian, cervical, and small cell lung cancer. Additional topo inhibitors and poisons continue to be developed and investigated.

The antibodies provided herein can be used to assess whether an enzyme-DNA complex including, for example, these covalent topo I-DNA complexes, have been stabilized in tumor cells after drug administration. As further described herein, such antibodies can be used for any appropriate immune-based assay (e.g., immunoblotting, immunofluorescence, or flow cytometry) to detect covalent enzyme-DNA complexes in, for example, intact tumor cells and tissues.

### Anti-Enzyme-Nucleic Acid Covalent Complex Antibodies

In one aspect, this disclosure provides an antibody having the ability to bind to enzyme-nucleic acid covalent complexes. The enzyme can be any appropriate enzyme including, without limitation, topo I (e.g., human topo I), topo II (e.g., human topo II), topo III, tyrosyl deoxynucleotidyl phosphodiesterase (TDP1), and yeast, protozoal, or bacterial topoisomerases (e.g., DNA gyrase, topoisomerase IV, and topoisomerase VI). The nucleic acid can be any type of nucleic acid including, without limitation, genomic DNA (e.g., genomic human DNA), plasmid DNA, or DNA from pathogenic organisms such as yeast, bacteria, or protozoa. In some cases, the antibodies provided herein can bind to human topo I-DNA covalent complexes.

In some embodiments, the K_{D} (the equilibrium dissociation constant) between an anti-enzyme-nucleic acid covalent complex antibody and an antigen will be no greater than 1×10⁻⁶, no greater than 1×10⁻⁷, no greater than 1×10⁻⁸, no greater than 1×10⁻⁹, no greater than 1×10⁻¹⁰, no greater than 1×10⁻¹¹, or no greater than 1×10⁻¹². In some embodiments, the antigen is an enzyme-nucleic acid covalent complex. In some embodiments, the antigen includes a polypeptide and a nucleoside, where the polypeptide and the nucleoside are covalently complexed. In some embodiments, the antigen includes a polypeptide and an oligonucleotide, where the polypeptide and the oligonucleotide are covalently complexed. In some embodiments, the K_{D} of an anti-enzyme-nucleic acid covalent complex antibody and an enzyme-nucleic acid covalent complex is less than the K_{D} of α-TopoI cc (disclosed in U.S. Pat. No. 8,530,172, which is incorporated herein by reference) and an enzyme-nucleic acid covalent complex.

Antibody selectivity, antibody affinity, and/or K_{D} may be calculated by methods known to a skilled artisan to quantify an antibody-antigen interaction including, for example, ELISA, competitive ELISA, Biacore or KinExA surface plasmon resonance analysis, and/or in vitro or in vivo neutralization assays, etc.

In some embodiments, the anti-enzyme-nucleic acid covalent complex antibody is able to detect enzyme-DNA covalent complexes in a cell after the cell is exposed to a drug treatment designed to affect enzyme-nucleic acid complexes. In some embodiments, the anti-enzyme-nucleic acid covalent complex antibody is able to detect presence, absence, and/or amount of enzyme-DNA covalent complex in a cell before, after, or before and after the cell is exposed to a drug treatment designed to affect enzyme-nucleic acid complexes. In some embodiments, the anti-enzyme-nucleic acid covalent complex antibody is able to detect presence, absence, and/or amount of enzyme-DNA covalent complex in a sample before, after, or before and after a subject from which the sample was obtained is exposed to a drug treatment designed to affect enzyme-nucleic acid complexes. In some embodiments, the subject is exposed to a drug treatment that targets topo I and/or topo II. In some embodiments, the anti-enzyme-nucleic acid covalent complex antibody is able to detect enzyme-DNA covalent complexes in a cell in vitro.

The antibodies provided herein can be any monoclonal or polyclonal antibody having specific binding affinity for an enzyme-nucleic acid covalent complex as opposed to the individual polypeptide subunit or the individual nucleic acid component. Such antibodies can be used in immunoassays in liquid phase or bound to a solid phase. For example, the antibodies provided herein can be used in competitive and non-competitive immunoassays in either a direct or indirect format. Examples of such immunoassays include the radioimmunoassay (MA), the enzyme-linked immunosorbent assay (ELISA), and the sandwich (immunometric) assay.

In some embodiments, the affinity of anti-enzyme-nucleic acid covalent complex antibody for an enzyme-nucleic acid covalent complex is at least 2 times, at least 3 times, at least 4 times, at least 5 times, at least 10 times, at least 15 times, at least 20 times, and least 30 times, at least 50 times, at least 75 times, or at least 100 times greater than the affinity of anti-enzyme-nucleic acid covalent complex antibody for the non-complexed enzyme.

In some embodiments, the affinity of anti-enzyme-nucleic acid covalent complex antibody for an enzyme-nucleic acid covalent complex is at least 2 times, at least 3 times, at least 4 times, at least 5 times, at least 10 times, at least 15 times, at least 20 times, and least 30 times, at least 50 times, at least 75 times, or at least 100 times greater than the affinity of anti-enzyme-nucleic acid covalent complex antibody for a peptide fragment of the non-complexed enzyme.

In some embodiments, the affinity of anti-enzyme-nucleic acid covalent complex antibody for a peptide fragment-nucleic acid covalent complex is at least 2 times, at least 3 times, at least 4 times, at least 5 times, at least 10 times, at least 15 times, at least 20 times, and least 30 times, at least 50 times, at least 75 times, or at least 100 times greater than the affinity of anti-enzyme-nucleic acid covalent complex antibody for the non-complexed peptide fragment.

In some embodiments, the affinity of anti-enzyme-nucleic acid covalent complex antibody for a peptide fragment-nucleic acid covalent complex is at least 2 times, at least 3 times, at least 4 times, at least 5 times, at least 10 times, at least 15 times, at least 20 times, and least 30 times, at least 50 times, at least 75 times, or at least 100 times greater than the affinity of anti-enzyme-nucleic acid covalent complex antibody for the non-complexed phosphorylated peptide fragment.

In some embodiments, the affinity of anti-enzyme-nucleic acid covalent complex antibody for an enzyme-nucleic acid covalent complex is at least 2 times, at least 3 times, at least 4 times, at least 5 times, at least 10 times, at least 15 times, at least 20 times, and least 30 times, at least 50 times, at least 75 times, or at least 100 times greater than the affinity of anti-enzyme-nucleic acid covalent complex antibody for the non-complexed nucleic acid.

In some embodiments, the affinity of anti-enzyme-nucleic acid covalent complex antibody for a peptide fragment-nucleic acid covalent complex is at least 2 times, at least 3 times, at least 4 times, at least 5 times, at least 10 times, at least 15 times, at least 20 times, and least 30 times, at least 50 times, at least 75 times, or at least 100 times greater than the affinity of anti-enzyme-nucleic acid covalent complex antibody for the non-complexed nucleic acid.

One can evaluate an antibody using any suitable method. For example, an antibody can be evaluated using, for example, ELISA to assess its ability to detect the antigen with the nucleoside compared to its ability to detect the antigen without the phosphorylated tyrosine or nucleoside. In some embodiments, an antibody can be evaluated to assess its ability to detect topo I-DNA complexes in lysates of cells treated with a topoisomerase inhibitor. In some cases, the antibody can detect topo I-DNA complexes in a lysate that is treated with a lower concentration of a topoisomerase inhibitor than is detectable using the antibody described in U.S. Pat. No. 8,530,172.

Antibodies provided herein can be prepared using any method. The preparation of polyclonal antibodies is well-known to those skilled in the art. See, e.g., Green et al., Production of Polyclonal Antisera, in IMMUNOCHEMICAL PROTOCOLS (Manson, ed.), pages 1-5 (Humana Press 1992) and Coligan et al., Production of Polyclonal Antisera in Rabbits, Rats, Mice and Hamsters, in CURRENT PROTOCOLS IN IMMUNOLOGY, section 2.4.1 (1992). In addition, those of skill in the art will know of various techniques common in the immunology arts for purification and concentration of polyclonal antibodies, as well as monoclonal antibodies (Coligan et al., Unit 9, Current Protocols in Immunology, Wiley Interscience, 1994).

The preparation of monoclonal antibodies also is well-known to those skilled in the art. See, e.g., Kohler & Milstein, Nature 1975, 256:495; Coligan et al., sections 2.5.1-2.6.7; and Harlow et al., ANTIBODIES: A LABORATORY MANUAL, page 726 (Cold Spring Harbor Pub. 1988). Briefly, monoclonal antibodies can be obtained by injecting mice with a composition including an antigen, verifying the presence of antibody production by analyzing a serum sample, removing the spleen to obtain B lymphocytes, fusing the B lymphocytes with myeloma cells to produce hybridomas, cloning the hybridomas, selecting positive clones that produce antibodies to the antigen, and isolating the antibodies from the hybridoma cultures. Monoclonal antibodies can be isolated and purified from hybridoma cultures by a variety of well-established techniques. Such isolation techniques include affinity chromatography with Protein A Sepharose, size exclusion chromatography, and ion exchange chromatography. See, e.g., Coligan et al., sections 2.7.1-2.7.12 and sections 2.9.1 2.9.3; Barnes et al., Purification of Immunoglobulin G (IgG), in METHODS IN MOLECULAR BIOLOGY, VOL. 10, pages 79-104 (Humana Press 1992).

In addition, methods of in vitro and in vivo multiplication of monoclonal antibodies are well known to those skilled in the art. Multiplication in vitro can be carried out in suitable culture media such as Dulbecco's Modified Eagle Medium or RPMI 1640 medium, optionally replenished by mammalian serum such as fetal calf serum, or trace elements and growth sustaining supplements such as normal mouse peritoneal exudate cells, spleen cells, and bone marrow macrophages. Production in vitro provides relatively pure antibody preparations and allows scale up to yield large amounts of the desired antibodies. Large scale hybridoma cultivation can be carried out by homogenous suspension culture in an airlift reactor, in a continuous stirrer reactor, or in immobilized or entrapped cell culture. Multiplication in vivo may be carried out by injecting cell clones into mammals histocompatible with the parent cells (e.g., syngeneic mice) to cause growth of antibody producing tumors. Optionally, the animals are primed with a hydrocarbon, especially oils such as pristane (tetramethylpentadecane) prior to injection. After one to three weeks, the desired monoclonal antibody is recovered from the body fluid of the animal.

An antibody fragment can be prepared by proteolytic hydrolysis of an intact antibody or by the expression of a nucleic acid encoding the fragment. An antibody fragment can be obtained by pepsin or papain digestion of an intact antibody by conventional methods. For example, an antibody fragment can be produced by enzymatic cleavage of an antibody with pepsin to provide a 5S fragment denoted F(ab′)2. This fragment can be further cleaved using a thiol reducing agent, and optionally a blocking group for the sulfhydryl groups resulting from cleavage of disulfide linkages, to produce 3.5S Fab′ monovalent fragments. In some cases, an enzymatic cleavage using pepsin can be used to produce two monovalent Fab′ fragments and an Fc fragment directly. These methods are described, for example, by Goldenberg (U.S. Pat. No. 4,036,945 and U.S. Pat. No. 4,331,647). See, also, Nisonhoff et al., Arch. Biochem. Biophys. 1960, 89:230 (1960); Porter. Biochem. J. 1959, 73:119; Edelman et al., METHODS IN ENZYMOLOGY, VOL 1, page 422 (Academic Press 1967); and Coligan et al. at sections 2.8,1-2,8.10 and 2.10.1-2.10.4.

Other methods of cleaving antibodies, such as separation of heavy chains to form monovalent light heavy chain fragments, further cleavage of fragments, or other enzymatic, chemical, or genetic techniques may also be used to provide fragments that retain some ability to bind (e.g., selectively bind) its epitope.

The antibodies provided herein can be substantially pure. The term “substantially pure” as used herein with reference to an antibody means the antibody is substantially free of other polypeptides, lipids, carbohydrates, and nucleic acids with which it is naturally associated in nature. Thus, a substantially pure antibody is any antibody that is removed from its natural environment and is at least 60 percent pure. A substantially pure antibody can be at least about 65, 70 75, 80 85, 90, 95, or 99 percent pure.

### Antigens Used to Generate Anti-Enzyme-Nucleic Acid Covalent Complex Antibodies

This disclosure further provides an antigen that includes a polypeptide and a nucleoside. In some embodiments, the polypeptide and the nucleoside are covalently complexed. In some embodiments, an antibody that binds to enzyme-nucleic acid covalent complexes can be generated using an antigen that includes a polypeptide and a nucleoside. In some embodiments, the polypeptide includes the active site peptide sequence of an enzyme that binds to DNA. In many embodiments, this active site includes a tyrosine. In some embodiments, the polypeptide includes the active site of a topoisomerase. In some embodiments, the polypeptide can include the active site of a topoisomerase and a terminal cysteine such as an N-terminal cysteine. In some embodiments, the N-terminal cysteine may be used to conjugate a covalent peptide-nucleotide complex to a carrier protein. In some embodiments, the polypeptide includes the active site of, for example, topo I, topo II, topo III, or TDP 1. In some embodiments, the antigen may include a polypeptide having or including a sequence set forth in Table 1.

TABLE 1

| | | |
|---|---|---|
| SEQ ID NO: 1 | CLGTSKLN | phospho-Topo I |
| | (phosphoY) | active site + N- |
| | LDPRITV | terminal cysteine |
| SEQ ID NO: 2 | CLGTSKLNYLDPRITV | Topo I active |
| | | site + N-terminal |
| | | cysteine |
| SEQ ID NO: 3 | CX₇(phospho-Y)X₇, | |
| | where X is a | |
| | random mixture | |
| | of amino acids | |
| SEQ ID NO: 4 | CKDSASPR | phospho-Topo IIα |
| | (phosphoY) | active site + N- |
| | IFTMLSS | terminal cysteine |
| SEQ ID NO: 5 | CKDAASPR | phospho-Topo IIβ |
| | (phosphoY) | active site + N- |
| | IFTMLST | terminal cysteine |
| SEQ ID NO: 6 | CKDSASPRYIFYMLSS | Topo IIα active |
| | | site + N-terminal |
| | | cysteine |
| SEQ ID NO: 7 | CKDAASPRYIFTMLST | Topo IIβ active |
| | | site + N-terminal |
| | | cysteine |
| SEQ ID NO: 11 | LGTSKLN | phospho-Topo I |
| | (phosphoY) | active site |
| | LDPRITV | |
| SEQ ID NO: 12 | LGTSKLNYLDPRITV | Topo I active site |
| SEQ ID NO: 13 | X₇(phospho-Y)X₇, | |
| | where X is a | |
| | random mixture | |
| | of amino acids | |
| SEQ ID NO: 14 | KDSASPR | phospho-Topo IIα |
| | (phosphoY) | active site |
| | IFTMLSS | |
| SEQ ID NO: 15 | KDAASPR | phospho-Topo IIβ |
| | (phosphoY) | active site |
| | IFTMLST | |
| SEQ ID NO: 16 | KDSASPRYIFTMLSS | Topo IIα active |
| | | site |
| SEQ ID NO: 17 | KDAASPRYIFTMLST | Topo IIβ active |
| | | site |

A nucleoside of an antigen can be covalently complexed to a polypeptide. In some embodiments, the nucleoside can be covalently complexed to a tyrosine. In some embodiments, the nucleoside is covalently complexed to an amino acid via, for example, a phosphate group, a phosphonate linkage, a carbon chain, etc. For example, a nucleoside can be covalently complexed to a polypeptide via a phosphorylated tyrosine in the polypeptide. In some embodiments, the tyrosine can be covalently linked to a nucleoside by a phosphate linkage to a 5′-alcohol. In some embodiments, the tyrosine can be covalently linked to a nucleoside by a phosphate linkage to a 3′-alcohol. The nucleoside may be any nucleoside. In some embodiments, the nucleoside may, for example, cytidine, deoxycytidine, uridine, adenosine, deoxyadenosine, guanosine, deoxyguanosine, thymidine, deoxythymidine, inosine, etc. In some embodiments, the nucleoside is a universal nucleoside. In some embodiments, the antigen may include multiple nucleosides and/or multiple nucleobases, for example, a homologate. In some embodiments where the antigen includes multiple nucleosides and/or multiple nucleobases, the nucleosides and/or nucleobases are connected to the same amino acid. In some embodiments where the antigen includes multiple nucleosides and/or multiple nucleobases, the nucleosides may be covalently complexed, for example, through a 3′ to 5′ phosphodiester linkage. In some embodiments where the antigen includes multiple nucleosides and/or multiple nucleobases, more than one nucleotide may be covalently complexed to the same amino acid, for example, a tyrosine, via a phosphate linkage. In some embodiments where the antigen includes multiple nucleosides and/or multiple nucleobases, the nucleosides and/or nucleobases are the same nucleotide or nucleobase; in other embodiments, the nucleosides and/or nucleobases are the different nucleotides or nucleobases.

In some embodiments, the antigen may include the structure 8, where B is a nucleobase. The nucleobase may be, for example, cytosine, guanine, adenine, thymine, uracil, pseudouridine, dihydrouridine, inosine, 7-methylguanosine, etc. In some embodiments, the nucleobase of structure 8 may be thymine, as shown in structure 9.

In some embodiments, the nucleoside may be modified. The nucleic acid may be modified as in, for example, hydroxymethylcytosine, methylcytosine, etc. In some embodiments, the sugar of the nucleoside may be modified. In some embodiments, the sugar of the nucleoside may include one or more R groups at the 2′ position, where the R groups can include an —OH group, a halogen; an alkyl group including, for example, methane, ethane, propane, and butane; a heteroalkyl group, etc. In some embodiments, the sugar of the nucleoside may be modified at the 5′ position with, for example, a triphosphate group, a phosphonate group, etc. In some embodiments, the nucleoside may be an acyclic nucleoside analog or an acyclic nucleoside phosphonate. In some embodiments, the antigen may include a nucleotide phosphoramidite, for example, a 2′-deoxynucleoside phosphoramidites. In some embodiments, a phosphoramidite is covalently complexed to a tyrosine.

In some embodiments, the antigen that includes a polypeptide and a nucleoside may further include an immunoadjuvant. An immunoadjuvant may include, for example, keyhole limpet hemocyanin (KLH); thyroglobulin, bovine serum albumin (BSA), tetanus toxoid, flagellin, a lipopeptide, a TLR agonist, an imidazoquinolone amine, and/or CpG, etc. In some embodiments, the immunoadjuvant may be chemically coupled to the polypeptide.

Methods for Making Antigens that Include a Polypeptide and a Nucleoside

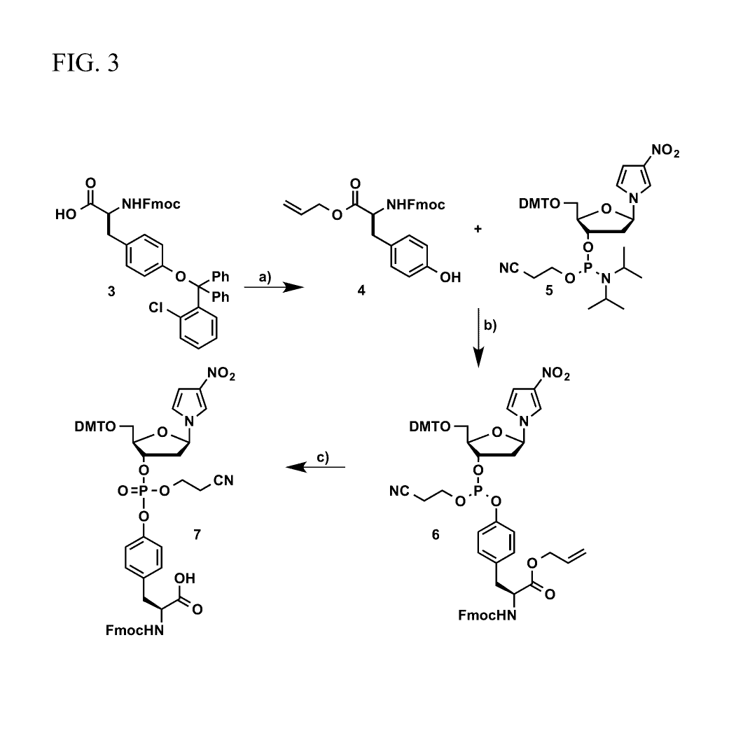

This disclosure also provides methods for generating compositions that include a polypeptide and a nucleoside and/or an antigen that includes a polypeptide and a nucleoside. Such compositions may be used to generate anti-enzyme-nucleic acid covalent complex antibodies. In some embodiments, for example wherein the polypeptide and the nucleoside are covalently complexed, the nucleoside portion of the antigen may be covalently linked to a tyrosine to form a nucleoside-tyrosine monomer. In some embodiments, the nucleoside portion of the antigen may be covalently linked to a tyrosine through a phosphate group to form a nucleoside-tyrosine monomer. The nucleoside-tyrosine monomer can be synthesized from an orthogonally protected tyrosine using the synthesis described in FIG. 3 and/or Smit et al. Angew Chem Int Ed Engl 2011, 50:9200-4. The method involves the synthesis of an amino acid including a tyrosine where the tyrosine is covalently complexed to the nucleoside by a phosphate linkage using a nucleoside phosphoramidite for coupling to the tyrosine followed by oxidation of the phosphite to the phosphate. In some embodiments, the tyrosine can be covalently linked to a nucleoside by a phosphate linkage to a 5′-alcohol. In some embodiments, the tyrosine can be covalently linked to a nucleoside by a phosphate linkage to a 3′-alcohol.

With this monomer prepared, the peptide can then be synthesized using standard solid-phase peptide synthesis. In some embodiments, the peptide portion of the antigen can be synthesized using standard 9-fluorenylmethyloxycarbonyl (Fmoc) solid phase peptide synthesis (SPPS) methodology. In some embodiments, the peptide can be synthesized on solid-phase resins using peptide coupling chemistry known to those skilled in the art, including, for example, rink amide resin using HBTU and N-methylmorpholine. In some embodiments, the peptide can be cleaved off resin using, for example, a cleavage cocktail of TFA:TIS:H₂O:EDT (85:5:5:5). In some embodiments, the peptide may be used without further processing. In some embodiments, the crude peptide may be concentrated; purified by HPLC; purified by chromatography methods including, for example, deactivated silica or ion-exchange chromatography; and/or isolated via precipitation, including for example, using cold ether. The resulting peptide can be purified, for example, by semi-preparative reverse-phase HPLC. The resulting peptide can be characterized, for example, by reverse-phase HPLC isolation and mass spectrometry (MS) analysis, tandem HPLC-MS, NMR spectroscopy, etc.

### Methods for Determining the Presence, Absence, or Amount of Enzyme-Nucleic Acid Complexes

This disclosure also provides methods for determining the presence, absence, or amount of enzyme-nucleic acid complexes (e.g., human topoisomerase I-DNA covalent complexes) within a sample (e.g., a biological sample such as a tissue biopsy). In some embodiments, the sample is a cell. Such methods include using an antibody provided herein in an immune-based assay to detect the presence, absence, and/or amount of enzyme-nucleic acid complexes within a sample obtained from a mammal. Any appropriate sample can be used. For example, tissue biopsies obtained from a hematological malignancy (e.g., leukemia and lymphoma), a solid tumor (e.g., non-small cell lung cancer, small cell lung cancer, colon cancer, and ovarian cancer), and/or a tissue harboring infectious organism containing topoisomerase can be obtained from a mammal and assessed for enzyme-nucleic acid complexes. In some cases, a sample of circulating tumor cells, cellular lysate, or partially purified enzyme sample can be assessed for enzyme-nucleic acid complexes. A sample to be assessed for enzyme-nucleic acid complexes can be obtained from any mammal including, without limitation, humans, non-human primates, dogs, rats, and/or mice. Any appropriate immune-based assays can be used to detect the presence, absence, and/or amount of enzyme-nucleic acid complexes within a sample. For example, immunoblotting, immunofluorescence, flow cytometry, ELISA, and/or immunohistochemistry can be used to detect covalent topo I-DNA complexes.

Methods for Monitoring the Effectiveness of a Drug Treatment Designed to Result in the Accumulation of Enzyme-Nucleic Acid Complexes within Cells

This disclosure also provides methods for monitoring the effectiveness of a drug treatment designed to result in the accumulation of enzyme-nucleic acid complexes (e.g., human topoisomerase I-DNA covalent complexes) within cells. For example, a mammal (e.g., a human) with cancer treated with TPT, camptothecin, or irinotecan can be assessed using the antibodies provided herein to determine if cells within the mammal accumulate enzyme-nucleic acid complexes. The accumulation of enzyme-nucleic acid complexes following drug treatment can indicate that the treatment is effective. In such cases, the mammal can be instructed to continue the treatment. A lack of accumulated enzyme-nucleic acid complexes following drug treatment can indicate that the treatment is not effective. In such cases, the mammal can be instructed to adjust or discontinue the treatment.

For example, in one embodiment, a sample may be obtained from a mammal treated with a drug treatment designed to result in the accumulation of enzyme-nucleic acid complexes, and an immune-based assay used to detect the presence, absence, and/or amount of enzyme-nucleic acid complexes within the sample. In some embodiments, the immune-based assay is preferably ELISA.

In some cases, the methods and materials provided herein can be used to assist medical or research professionals in determining whether or not a particular drug treatment is effective. Medical professionals can be, for example, doctors, nurses, medical laboratory technologists, and pharmacists. Research professionals can be, for example, principle investigators, research technicians, postdoctoral trainees, and graduate students. A professional can be assisted by (1) determining whether or not a mammal (e.g., a human) accumulates enzyme-nucleic acid complexes (e.g., human topoisomerase I-DNA covalent complexes) within cells following treatment, and (2) communicating information about the presence or absence of such an accumulation to that professional.

Any appropriate method can be used to communicate information to another person (e.g., a professional). For example, information can be given directly or indirectly to a professional. In addition, any type of communication can be used to communicate the information. For example, mail, e-mail, telephone, and face-to-face interactions can be used. The information also can be communicated to a professional by making that information electronically available to the professional. For example, the information can be communicated to a professional by placing the information on a computer database such that the professional can access the information. In addition, the information can be communicated to a hospital, clinic, or research facility serving as an agent for the professional.

### Exemplary Embodiments

Embodiment 1. An isolated antibody preparation comprising an anti-enzyme-nucleic acid covalent complex antibody, wherein the anti-enzyme-nucleic acid covalent complex antibody binds to an enzyme-nucleic acid covalent complex and does not bind to the free form of the enzyme or the free form of the nucleic acid, and wherein the anti-enzyme-nucleic acid covalent complex antibody is produced using an antigen that comprises a polypeptide and a nucleoside, wherein the polypeptide and the nucleoside are covalently complexed.
Embodiment 2. An isolated antibody preparation comprising an anti-enzyme-nucleic acid covalent complex antibody, wherein the anti-enzyme-nucleic acid covalent complex antibody binds to an enzyme-nucleic acid covalent complex and does not bind to the free form of the enzyme or the free form of the nucleic acid, and wherein the anti-enzyme-nucleic acid covalent complex antibody binds to an antigen that comprises a polypeptide and a nucleoside, wherein the polypeptide and the nucleoside are covalently complexed.
Embodiment 3. The isolated antibody preparation of either of Embodiments 1 or 2, wherein the nucleic acid is DNA.
Embodiment 4. The isolated antibody preparation of any of Embodiments 1 to 3, wherein the polypeptide comprises a phosphorylated tyrosine, and further wherein the nucleoside is covalently complexed to the phosphorylated tyrosine.
Embodiment 5. The isolated antibody preparation of any of Embodiments 1 to 4, wherein the nucleoside is covalently complexed to the phosphorylated tyrosine by a phosphate linkage to a 5′-alcohol or a 3′-alcohol.
Embodiment 6. The isolated antibody preparation of any of Embodiments 1 to 5, wherein the polypeptide comprises the amino acid sequence set forth in SEQ ID NO:1, SEQ ID NO:4, SEQ ID NO:5, SEQ ID NO:11, SEQ ID NO:13, or SEQ ID NO:14.
Embodiment 7. The isolated antibody preparation of any of Embodiments 1 to65, wherein the nucleoside comprises at least one of cytidine, deoxycytidine, uridine, deoxyuridine, adenosine, deoxyadenosine, guanosine, deoxyguanosine, thymidine, or deoxythymidine.
Embodiment 8. The isolated antibody preparation of any of Embodiments 1 to 7, wherein the antigen comprises SEQ ID NO:9 (structure 8).
Embodiment 9. The isolated antibody preparation of any of Embodiments 1 to 8, wherein the antigen comprises SEQ ID NO:10 (structure 9).
Embodiment 10. An isolated antibody preparation comprising an anti-enzyme-nucleic acid covalent complex antibody, wherein the anti-enzyme-nucleic acid covalent complex antibody binds to an enzyme-nucleic acid covalent complex and does not bind to the free form of the enzyme or the free form of the nucleic acid.
Embodiment 11. The isolated antibody preparation of any of Embodiments 1 to 10, wherein the K_{D} of the anti-enzyme-nucleic acid covalent complex antibody and an enzyme-nucleic acid covalent complex is no greater than 1×10⁻⁸.
Embodiment 12. The isolated antibody preparation of any of Embodiments 1 to 11, wherein the K_{D} of the anti-enzyme-nucleic acid covalent complex antibody and an enzyme-nucleic acid covalent complex is no greater than 1×10⁻⁹.
Embodiment 13. The isolated antibody preparation of any of Embodiments 1 to 12, wherein the K_{D} of the anti-enzyme-nucleic acid covalent complex antibody and an enzyme-nucleic acid covalent complex is no greater than 1×10⁻¹⁰.
Embodiment 14. The isolated antibody preparation of any of Embodiments 1 to 13, wherein the K_{D} of the anti-enzyme-nucleic acid covalent complex antibody and an enzyme-nucleic acid covalent complex is no greater than 1×10⁻¹¹.
Embodiment 15. The isolated antibody preparation of any of Embodiments 1 to 14, wherein the K_{D} of the anti-enzyme-nucleic acid covalent complex antibody and an enzyme-nucleic acid covalent complex is no greater than 1×10⁻¹².
Embodiment 16. A method for assessing a sample for enzyme-nucleic acid covalent complexes, wherein the method comprises performing an immune-based assay using an anti-enzyme-nucleic acid covalent complex antibody to detect the presence, absence, or amount of the enzyme-nucleic acid covalent complexes within the sample, wherein the anti-enzyme-nucleic acid covalent complex antibody binds to the enzyme-nucleic acid covalent complex and does not bind to the free form of the enzyme or the free form of the nucleic acid, and further wherein the K_{D} of the anti-enzyme-nucleic acid covalent complex antibody and the enzyme-nucleic acid covalent complex is no greater than 1×10⁻⁸.
Embodiment 17. The method of Embodiment 16, wherein the K_{D} of the anti-enzyme-nucleic acid covalent complex antibody and the enzyme-nucleic acid covalent complex is no greater than 1×10⁻⁹.
Embodiment 18. The method of Embodiment 17, wherein the K_{D} of the anti-enzyme-nucleic acid covalent complex antibody and the enzyme-nucleic acid covalent complex is no greater than 1×10^{-10.}
Embodiment 19. The method of Embodiment 18, wherein the K_{D} of the anti-enzyme-nucleic acid covalent complex antibody and the enzyme-nucleic acid covalent complex is no greater than 1×10⁻¹¹.
Embodiment 20. The method of Embodiment 19, wherein the K_{D} of the anti-enzyme-nucleic acid covalent complex antibody and the enzyme-nucleic acid covalent complex is no greater than 1×10⁻¹².
Embodiment 21. The method of any of Embodiments 16 to 20, wherein the sample comprises a cell or a tissue sample.
Embodiment 22. The method of any of Embodiments 16 to 21, wherein the anti-enzyme-nucleic acid covalent complex antibody is able to detect the presence of the enzyme-nucleic acid covalent complexes within the sample, wherein the sample is obtained from a subject following administration to the subject of a drug treatment designed to result in the accumulation of enzyme-nucleic acid covalent complexes within cells.
Embodiment 23. A method for monitoring the effectiveness of a drug treatment designed to result in the accumulation of enzyme-nucleic acid covalent complexes within cells, wherein the method comprises performing an immune-based assay using an anti-enzyme nucleic acid covalent complex antibody to detect the presence or absence of the enzyme-nucleic acid covalent complexes within a sample obtained from a mammal following treatment of the mammal with the drug treatment, wherein the anti-enzyme-nucleic acid covalent complex antibody binds to the nucleic acid covalent complex and does not bond to the free form of the enzyme, and further wherein the K_{D} of the anti-enzyme-nucleic acid covalent complex antibody and the enzyme-nucleic acid covalent complex is no greater than 1×10⁻⁸.
Embodiment 24. The method of Embodiment 23, wherein the drug treatment is a topoisomerase I inhibitor or poison.
Embodiment 25. The method of either of Embodiments 23 or 24, wherein the drug treatment is an irinotecan, topotecan, or camptothecin treatment.
Embodiment 26. The method of Embodiment 23, wherein the drug treatment is a topoisomerase II inhibitor or poison.
Embodiment 27. A composition comprising a polypeptide and a nucleoside, wherein the polypeptide comprises the amino acid sequence of an active site of an enzyme that binds to DNA, and further wherein the polypeptide and the nucleoside are covalently complexed via a phosphate group.
Embodiment 28. The composition of Embodiment 27, wherein the polypeptide comprises the amino acid sequence set forth in SEQ ID NO:1, SEQ ID NO:4, SEQ ID NO:5, SEQ ID NO:11, SEQ ID NO:13, or SEQ ID NO:14.
Embodiment 29. The composition of either of Embodiments 27 or 28, wherein the nucleoside is cytidine, deoxycytidine, uridine, deoxyuridine, adenosine, deoxyadenosine, guanosine, deoxyguanosine, thymidine, or deoxythymidine.
Embodiment 30. The composition of any of Embodiments 27 to 29, wherein the enzyme that binds to DNA is topoisomerase I or topoisomerase II.
Embodiment 31. The composition of any of Embodiments 26 to 29, wherein the polypeptide comprises phosphorylated tyrosine, and further wherein the nucleoside is covalently complexed to the phosphorylated tyrosine.
Embodiment 32. The composition of embodiment 31, wherein the nucleoside is covalently complexed to the phosphorylated tyrosine by a phosphate linkage to a 5′-alcohol or a 3′-alcohol.
Embodiment 33. A composition comprising

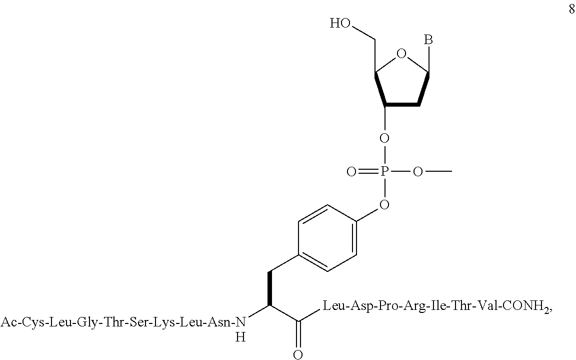

wherein B comprises a nucleobase.
Embodiment 34. A composition comprising

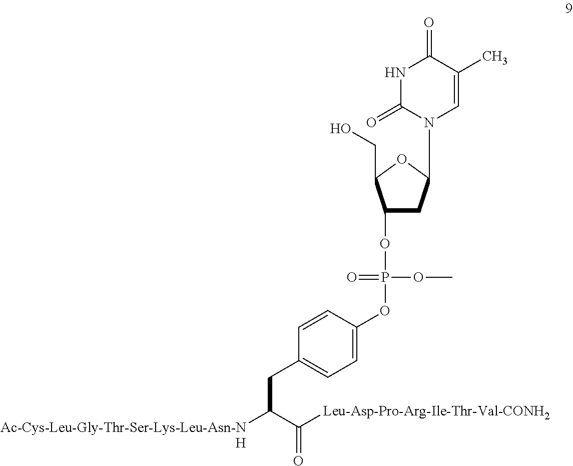

Embodiment 35. A method of making the composition of any of Embodiments 27 to 34.

The present invention is illustrated by the following examples. It is to be understood that the particular examples, materials, amounts, and procedures are to be interpreted broadly in accordance with the scope and spirit of the invention as set forth herein.

### EXAMPLES

### Example 1

Described in this example is the synthesis of a topo I-DNA covalent complex that contains a universal nucleoside. Portions of this Example have also been published in Perkins et al., Organic and Biomolecular Chemistry, 2016, 14:4103-9.

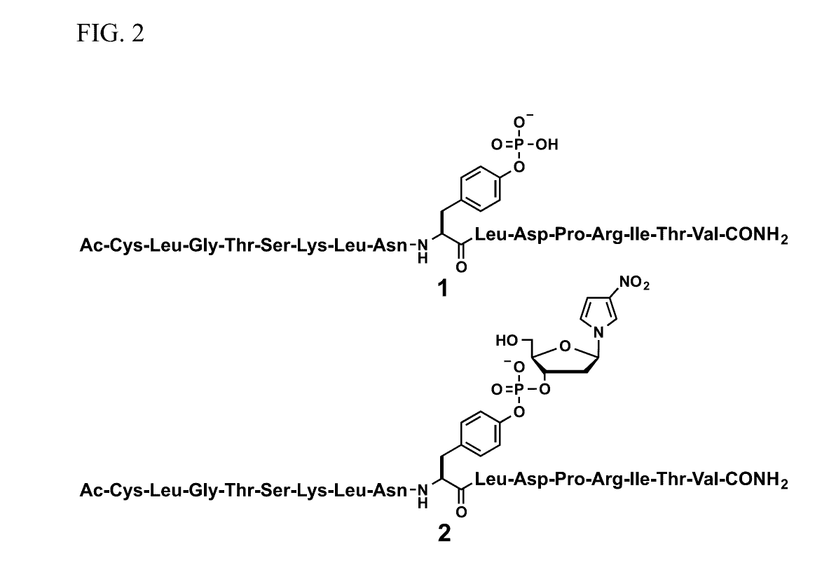

In certain applications, monoclonal antibody, α-TopoI cc, raised against antigen 1 (FIG. 2) (U.S. Pat. No. 8,530,172), can lack sufficient sensitivity and/or selectivity to detect therapeutically relevant levels of topo I-DNA covalent complex in patient samples (Patel et al., Nucleic Acids Research, 2016, 44(6):2816-26). This Example describes the preparation of peptide antigen 2 (FIG. 2), which mimics the topo I active site and contains a universal nucleoside, 1-ribofuranosyl-2′-deoxy-3-nitropyrrole (3-NP), appended to the catalytic tyrosine. In addition, the development of 5 monoclonal antibodies against antigen 2 and their characterization is reported.

Antigen 2, requires conjugation of the 3′-hydroxyl group of 3-NP to the catalytic tyrosine. To prepare antigen 2, the method of Itzen and Hedberg that requires synthesis of the fully modified tyrosine amino acid followed by its utilization in SPPS was adapted. (Smit et al. Angew Chem Int Ed Engl 2011, 50:9200-4; Hedberg, et al. ACS Chem Biol 2015, 10:12-21; Muller et al. ChemBioChem 2014, 15:19-26.) This approach began with synthesis of an orthogonally protected tyrosine followed by removal of the 2-chlorotrityl protecting group to afford known 3 (FIG. 3). (Ficht et al. Chem Eur J 2008, 14:3620-9.) Reaction of the free phenol 4 with commercially available phosphoramidite 5 yielded phosphite 6 in 69% yield. Oxidation of the phosphite to the phosphate was performed using conditions optimized for related compounds, resulting in clean formation of 6. (Smit et al. Angew Chem Int Ed Engl 2011, 50:9200-4.) Pd-catalyzed removal of the alloc protecting group provided target monomer 7 in 53% yield. (Dessolin et al. Tetrahedron Lett 1995, 36:5741-5744.)

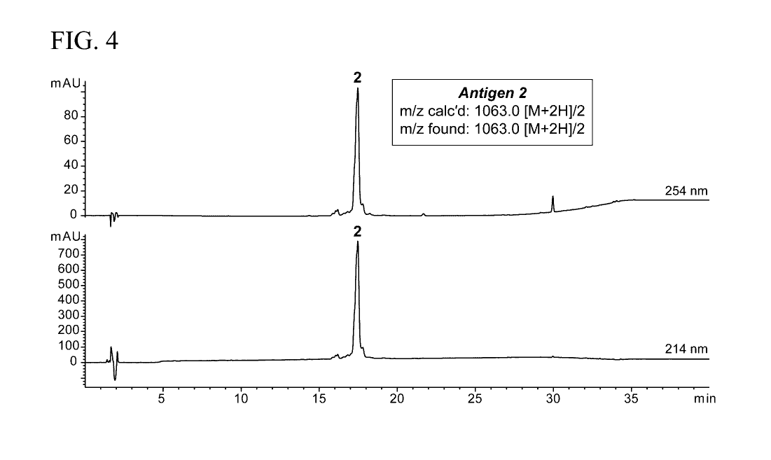

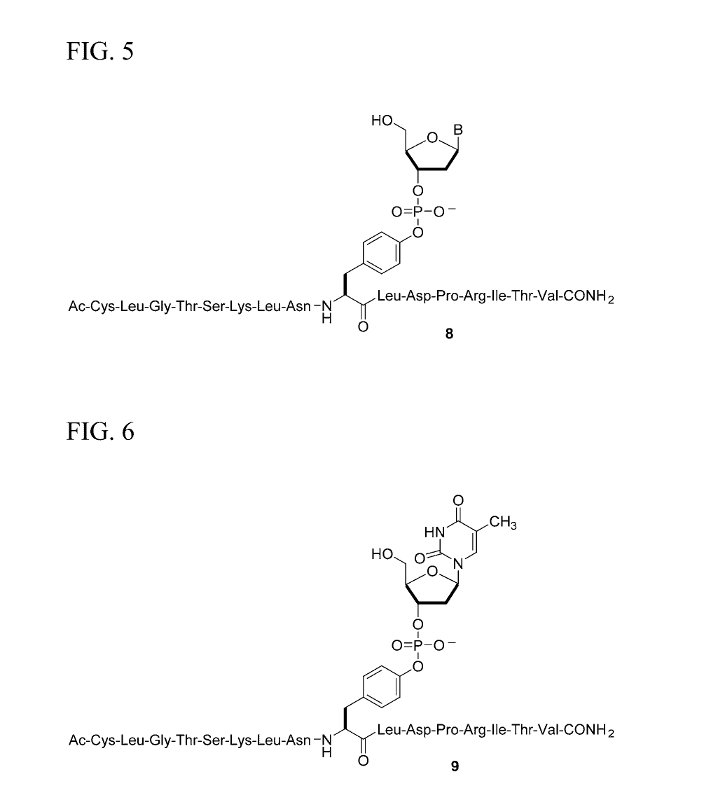

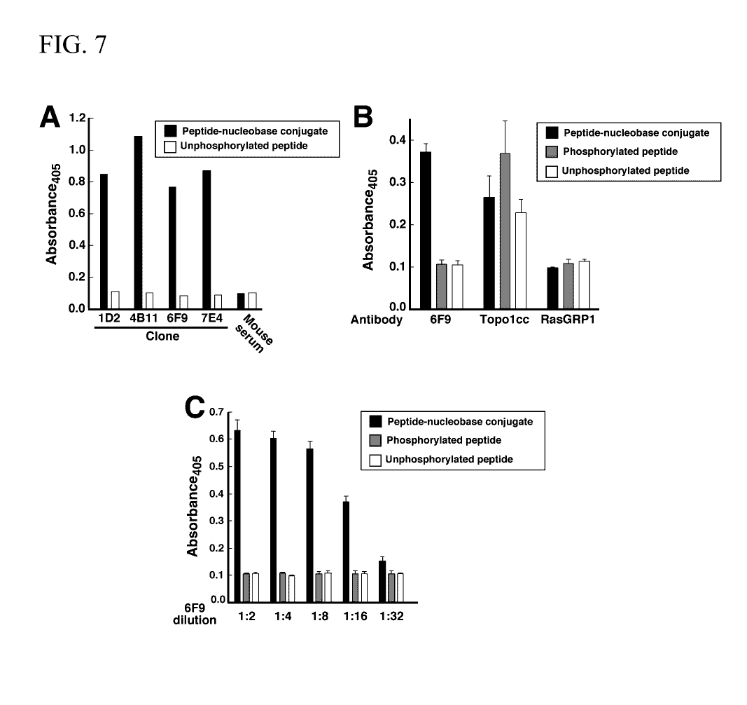

With monomer 7 complete, antigen 2 was synthesized using standard Fmoc SPPS methodology (Millipore, E. Peptide Synthesis Technical Resources). The peptide was built on rink amide resin using HBTU and N-methylmorpholine for couplings. The peptide was cleaved off resin using the cleavage cocktail of TFA:TIS:H₂O:EDT (85:5:5:5). The crude peptide was then isolated via precipitation using cold ether (-3x amount of cleavage cocktail) and the white precipitate was collected via centrifugation. Purification of the resulting peptide by semi-preparative reverse-phase HPLC and mass spectrometry characterization confirmed the synthesis of 2 in 6% isolated yield. An analytical HPLC chromatogram of purified 2 is shown in FIG. 4.

With antigen 2 in-hand, 5 monoclonal antibodies were raised. However, while the antibodies raised against antigen 2 could detect a topo I-DNA covalent complex that contains a universal nucleoside, the antibodies raised against antigen 2 failed to detect the covalent topo I-DNA complex found in patient samples. That is, this strategy yielded antibodies with apparent specificity for nucleosides bearing 3-nitropyrrole nucleobases.

### Example 2

Described in this example is a method for synthesizing a topo I-DNA covalent complex that contains a nucleoside.

Following synthesis of an orthogonally protected tyrosine, removal of the 2-chlorotrityl protecting group affords known 3 (FIG. 3). (Ficht et al. Chem Eur J2008, 14:3620-9.) Treatment of 3 with first, allyl bromide, DIPEA, DMF; then CF₃CO₂H in CH₂Cl₂ (1:49) (2 steps) yields the free phenol 4. Reaction of the free phenol 4 with a suitably protected nucleotide phosphoramidite (e.g. thymidine phosphoramidite, deoxythymidine phosphoramidite, adenine phosphoramidite, deoxyadenine phosphoramidite, guanine phosphoramidite, deoxyguanine phosphoramidite, cytidine phosphoramidite, deoxycytidine phosphoramidite, uracil phosphoramidite, deoxyuridine phosphoramidite, etc.) (in tetrazole (0.47M) in CH₂Cl₂, 4 Å mol. sieves) yields the corresponding phosphite. Oxidation of the phosphite to the phosphate is performed using conditions (t-BuOOH (5-6 M in decane), CH₂Cl₂) optimized for related compounds. (Smit et al. Angew Chem Int Ed Engl 2011, 50:9200-4.) Pd-catalyzed removal of the alloc protecting group (using Pd(PPh₃)₄, PhSiH₃, THF) provides the target monomer. (Dessolin et al. Tetrahedron Lett 1995, 36:5741-5744.)

The peptide portion of the antigen is synthesized using standard Fmoc SPPS methodology. (Millipore, E. Peptide Synthesis Technical Resources) The peptide is built on rink amide resin using HBTU and N-methylmorpholine for couplings. The peptide is cleaved off resin using a cleavage cocktail of TFA:TIS:H₂O:EDT (85:5:5:5). The crude peptide is then isolated via precipitation using cold ether (˜3× amount of cleavage cocktail) and the white precipitate is collected via centrifugation. The resulting peptide is purified by semi-preparative reverse-phase HPLC. Purity is checked by re-injection onto an analytical HPLC followed by MS characterization.

### Example 3

Monoclonal antibody generation. Using derivatized topo I peptide (including antigen 2) conjugated through an N-terminal cysteine to keyhole limpet hemocyanin as an antigen, murine hybridomas that detect the derivatized topo I peptide were generated and cloned as described by de St Groth et al. (J Immunol Methods 1980; 35(1-2):1-21). Primary screening of culture supernatants was performed by ELISA; and secondary screening was performed as described below. ELISA. Immobilon II ELISA plates (Thermo) were coated with 1 μg/well of the indicated peptide in 100 mM sodium carbonate buffer overnight at 4° C., washed with calcium- and magnesium-free Dulbecco's phosphate buffered saline (PBS), and blocked with 3% bovine serum albumin in PBS. Wells were incubated with concentrated culture supernatant from cloned hybridoma lines for 1.5-2 h, washed three times with PBS containing 0.05% Tween 20, incubated for alkaline phosphatase-conjugated goat anti-mouse IgG (Sigma-Aldrich) at 0.2 μg/ml for 1 h, washed four times with PBS containing 0.05% Tween 20, incubated for 5 min in 10 mM diethanolamine (pH 9.5) and then reacted with 1 mg/ml p-nitrophenyl phosphate in 10 mM diethanolamine for 12 min before absorbance was read at 405 nm.

### Example 4

Monoclonal antibody generation. Using peptide-nucleotide antigen 8 and/or peptide-nucleotide antigen 9, murine hybridomas are generated and cloned as described by de St Groth et al. (J Immunol Methods 1980; 35(1-2):1-21). Primary screening of culture supernatants is performed by ELISA; and secondary screening is performed as described below.

ELISA. Immobilon II ELISA plates (Thermo) are coated with 1 μg/well of the indicated peptide in 100 mM sodium carbonate buffer overnight at 4° C., washed with calcium- and magnesium-free Dulbecco's phosphate buffered saline (PBS), and blocked with 3% bovine serum albumin in PBS. Wells are incubated with concentrated culture supernatant from cloned hybridoma lines for 1.5-2 h, washed three times with PBS containing 0.05% Tween 20, incubated for alkaline phosphatase-conjugated goat anti-mouse IgG (Sigma-Aldrich) at 0.2 μg/ml for 1 h, washed four times with PBS containing 0.05% Tween 20, incubated for 5 min in 10 mM diethanolamine (pH 9.5) and then reacted with 1 mg/ml p-nitrophenyl phosphate in 10 mM diethanolamine for 12 min before absorbance is read at 405 nm.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

What is claimed is:

1. An isolated antibody preparation comprising an anti-enzyme-nucleic acid covalent complex antibody, wherein the anti-enzyme-nucleic acid covalent complex antibody binds to an enzyme-nucleic acid covalent complex and does not bind to the free form of the enzyme or the free form of the nucleic acid, and wherein the anti-enzyme-nucleic acid covalent complex antibody binds to an antigen that comprises a polypeptide and a nucleoside, wherein the polypeptide and the nucleoside are covalently complexed, and wherein a K_{D} of the anti-enzyme-nucleic acid covalent complex antibody and an enzyme-nucleic acid covalent complex is no greater than 1×10⁻⁸.

2. The isolated antibody preparation of claim 1, wherein the nucleic acid is DNA.

3. The isolated antibody preparation of claim 1, wherein the polypeptide comprises a phosphorylated tyrosine, and further wherein the nucleoside is covalently complexed to the phosphorylated tyrosine.

4. The isolated antibody preparation of claim 1, wherein the polypeptide comprises the amino acid sequence set forth in SEQ ID NO:1, SEQ ID NO:4, SEQ ID NO:5, SEQ ID NO:11, SEQ ID NO:13, or SEQ ID NO:14.

5. The isolated antibody preparation of claim 1, wherein the anti-enzyme-nucleic acid covalent complex antibody is produced using a polypeptide antigen comprising the amino acid sequence set forth in SEQ ID NO:1, SEQ ID NO:4, SEQ ID NO:5.

6. The isolated antibody preparation of claim 1, wherein the nucleoside comprises cytidine, deoxycytidine, uridine, deoxyuridine, adenosine, deoxyadenosine, guanosine, deoxyguanosine, thymidine, or deoxythymidine.

7. The isolated antibody preparation of claim 1, wherein the antigen comprises 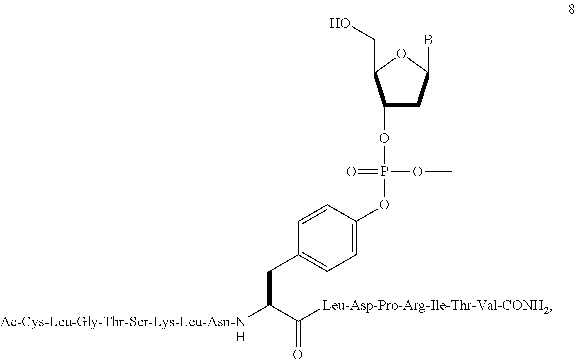
wherein B comprises a nucleobase.

8. The isolated antibody preparation of any of claims 1 to 6, wherein the antigen comprises

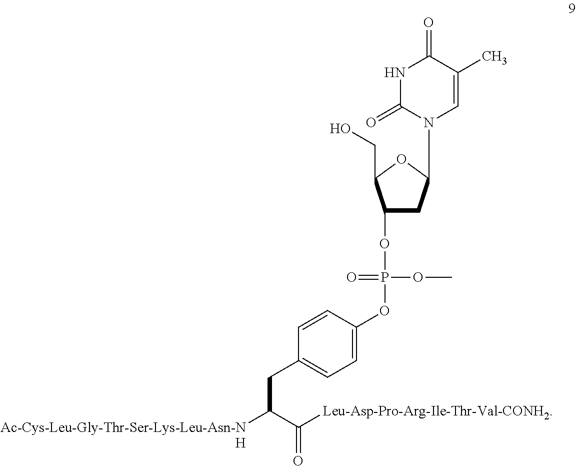

9. The isolated antibody preparation of claim 1, wherein the anti-enzyme-nucleic acid covalent complex antibody is produced using a polypeptide antigen comprising 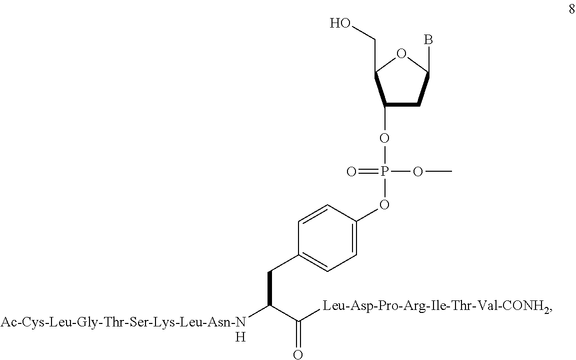
wherein B comprises a nucleobase.

10. The isolated antibody preparation of claim 1, wherein the K_{D} of the anti-enzyme-nucleic acid covalent complex antibody and an enzyme-nucleic acid covalent complex is no greater than 1×10⁻¹².

11. The isolated antibody preparation of claim 1, wherein the enzyme comprises a topoisomerase.

12. The isolated antibody preparation of claim 1, wherein the antibody is a monoclonal antibody.

13. A method for assessing a sample for enzyme-nucleic acid covalent complexes, wherein the method comprises performing an immune-based assay using an anti-enzyme-nucleic acid covalent complex antibody to detect the presence, absence, or amount of the enzyme-nucleic acid covalent complexes within the sample, wherein the anti-enzyme-nucleic acid covalent complex antibody binds to the enzyme-nucleic acid covalent complex and does not bind to the free form of the enzyme or the free form of the nucleic acid, and further wherein the K_{D} of the anti-enzyme-nucleic acid covalent complex antibody and the enzyme-nucleic acid covalent complex is no greater than 1×10⁻⁸.

14. The method of claim 13, wherein the sample comprises a cell or a tissue sample.

15. The method of claim 13, wherein the K_{D} of the anti-enzyme-nucleic acid covalent complex antibody and an enzyme-nucleic acid covalent complex is no greater than 1×10⁻¹².

16. The method of claim 13, wherein the anti-enzyme-nucleic acid covalent complex antibody is able to detect the presence of the enzyme-nucleic acid covalent complexes within the sample, wherein the sample is obtained from a subject following administration to the subject of a drug treatment designed to result in the accumulation of enzyme-nucleic acid covalent complexes within cells.

17. The method of claim 13, wherein the drug treatment is a topoisomerase I inhibitor or poison.

18. The method of claim 13, wherein the drug treatment is an irinotecan, topotecan, or camptothecin treatment.

19. The method of claim 13, wherein the drug treatment is a topoisomerase II inhibitor or poison.
